(19) United States
(12) Patent Application Publication
(10) Pub. No.: US 2019/0329005 A1
(43) Pub. Date: Oct. 31, 2019

(54) CATHETER SECURING WRAP

(71) Applicant: Jenny Ishii, Malden, MA (US)

(72) Inventor: Jenny Ishii, Malden, MA (US)

(21) Appl. No.: 15/966,766

(22) Filed: Apr. 30, 2018

(51) Int. Cl. A61M 25/02

(52) U.S. Cl. CPC A61M 25/02; A61M 2025/0246; A61M 2025/0266; A61M 2025/026

(57) ABSTRACT

A securement wrap for a catheter and other devices includes a wrap portion having a top side, a bottom side, a first end, a second end, and a central area located between the first and second ends. The wrap portion formed of an elastic self-adherent material and includes an adhesive layer comprising first and second adhesive portions disposed at each of the first and second ends of the bottom side of the wrap portion, respectively. The first and second adhesive portions for attaching the respective first and second ends of the wrap portion to the skin of a person. The central portion being releasable attachable to a catheter or other device for securing the catheter or other device to the body of a person.

### FIELD OF THE INVENTION

The present invention relates generally to medical catheters, and more particularly to a securing wrap for catheters and other devices.

### BACKGROUND OF THE INVENTION

The statements in this section merely provide background information related to the present disclosure and may not constitute prior art.

Many modern medical devices and procedures utilize catheters which are inserted into a human body through an opening, such as a urinary tract, a blood vessel, or other opening and are then routed to a desired location for the performance of a diagnostic or therapeutic procedure. The end of the catheter located outside of the body may include a coupler for connecting the catheter to another device for various purposes. For example, the catheter may be a urinary catheter which may be releasably connectable to a drainage bag or a drainage tube for removing urine from the body. In other applications, the catheter may be used as a pathway for inserting fluids or devices into the body. As the procedures and treatments Which can be carried out by means of such catheters have become more numerous, the use of catheters in medical procedures and therapies is increasing.

Often it may be advantageous to leave the catheter inserted into an opening in the body for extended periods. When not in use, the end of the catheter outside of the body is often fixed to the body with surgical tape or the like, to retain the catheter against the body in a secure position. Typically, once the procedure is complete, or o change or re-secure the catheter against the body, it may be necessary to remove the tape from the catheter. Often, the tape may be hard to remove from the catheter and may leave sticky adhesive residue on the catheter. Adhesive residue left on the catheter may attract dirt and often discolors the catheter.

Other devices such as tubes for insulin pumps and glucose monitoring devices for example, may also need to be secured to a person's body for extended periods of time. Typically, most of these devices are formed of plastics, wherein tape may leave a sticky residue on the devices which collect dirt and may be difficult to remove.

Accordingly, it is an object of the present teachings to provide a securement wrap for catheters and other devices that overcomes the shortcomings of the prior art.

### SUMMARY OF THE INVENTION

This section provides a general summary of the disclosure and does provide a comprehensive description or include full scope or all the features of the subject matter disclosed.

According to one aspect, the present teachings provides a securement wrap for a catheter and other devices including a wrap portion having a top side, a bottom side, a first end, a second end, and a central area located between the first and second ends. The wrap portion formed of an elastic self-adherent material and includes an adhesive layer comprising first and second adhesive portions disposed at each of the first and second ends of the bottom side of the wrap portion, respectively. The first and second adhesive portions for attaching the respective first and second ends of the wrap portion to the skin of a person. The central portion being releasable attachable to a catheter or other device for securing the catheter or other device to the body of a person.

In another aspect, a securement wrap is provided for a catheter and other devices including a wrap portion having a top side, a bottom side, a first end, and a second end. The wrap portion formed of an elastic self-adherent material and includes an adhesive layer comprising an adhesive portion disposed at the first end of the bottom side of the wrap portion. The adhesive portion for attaching the first end of the wrap portion to the skin of a person. The wrap being configured to encircle a catheter or other device and secure the same to the body of a patient.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The present teachings will become more fully understood from the detailed description, the appended claims and the following drawings. The drawings are for illustrative purposes only and are not intended to limit the scope of the present disclosure.

FIG. 1 is a perspective view of one embodiment of a securement wrap in accordance with the present invention.

FIG. 2 is a perspective view of a bottom side of the securement wrap of FIG. 1 including protective tapes attached to the securement wrap.

FIG. 3 is a perspective view of the bottom side of the securement wrap of FIG. 1 showing adhesive portions of the securement wraps without the protective tapes.

FIG. 4 is a perspective view of the securement wrap of FIG. 1 shown as used with a catheter.

FIG. 5 is a perspective view of another embodiment of a securement wrap in accordance with the present invention.

FIG. 6 is a perspective view of a bottom side of the securement wrap of FIG. 5 including protective tape attached to the securement wrap.

FIG. 7 is a perspective view of the bottom side of the securement wrap of FIG. 5 showing an adhesive portion of the securement wraps without the protective tape.

FIG. 8 is a perspective view of the securement wrap of FIG. 5 shown as used with a catheter.

FIG. 9 is a perspective view of the securement wraps of FIG. 1 and FIG. 5 shown as used with a catheter secured to a patient's body.

### DETAILED DESCRIPTION OF EMBODIMENTS OF THE INVENTION

Detailed illustrative descriptions of example embodiments are disclosed herein. However, specific structural and functional details disclosed herein are merely representative for purposes of describing example embodiments. The example embodiments may be embodied in many alternate forms and should not be construed as limited to only the example embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term “and/or” includes any and all combinations of one or more of the associated listed items.

It will be understood that when an element is referred to as being “connected,” “coupled,” “mated,” “attached,” or “fixed” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between”versus “directly between”, “adjacent” versus “directly adjacent”, etc.).

The terminology used herein is for the purpose of describing particular embodiments only and is not intended to be limiting of example embodiments. As used herein, the singular forms “a”, “an” and “the” are intended to include the plural forms as well, unless the language explicitly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

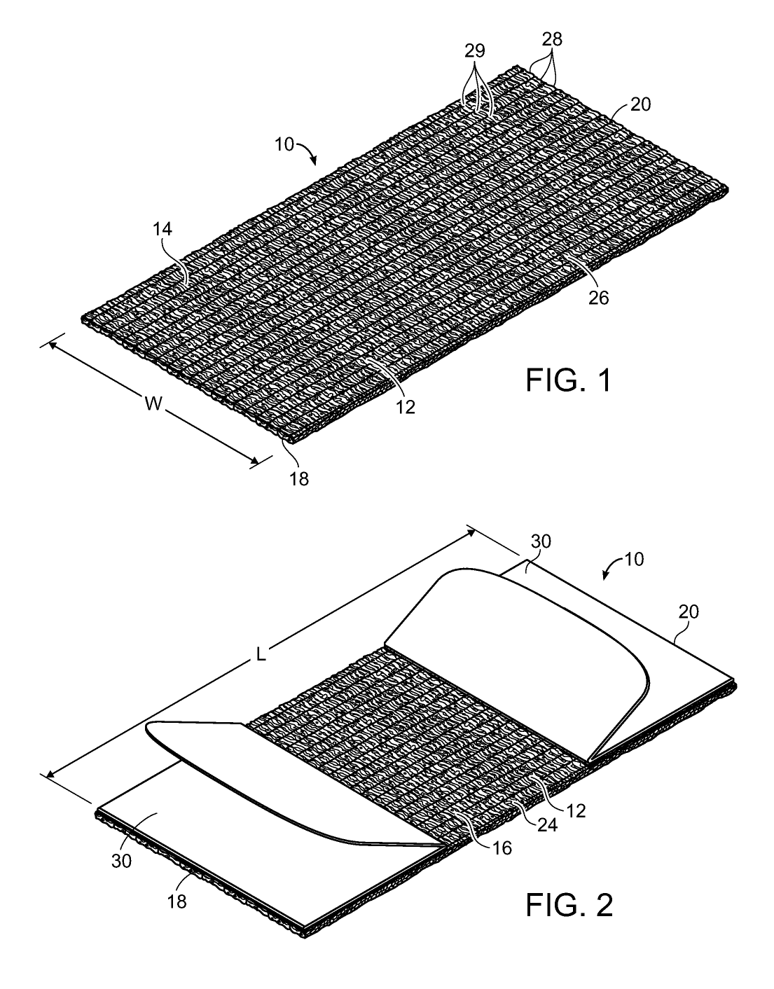

FIGS. 1-4 show a securement wrap 10 according to the present disclosure, including a wrap portion 12 having a top side 14, a bottom side 16, a first end 18, a second end 20, and a central area 24 located between the first and second ends. In the illustrated embodiment, the wrap portion 12 is formed of an elastic self-adherent material 26. The material 26 being flexible and conformable to a body part or other object.

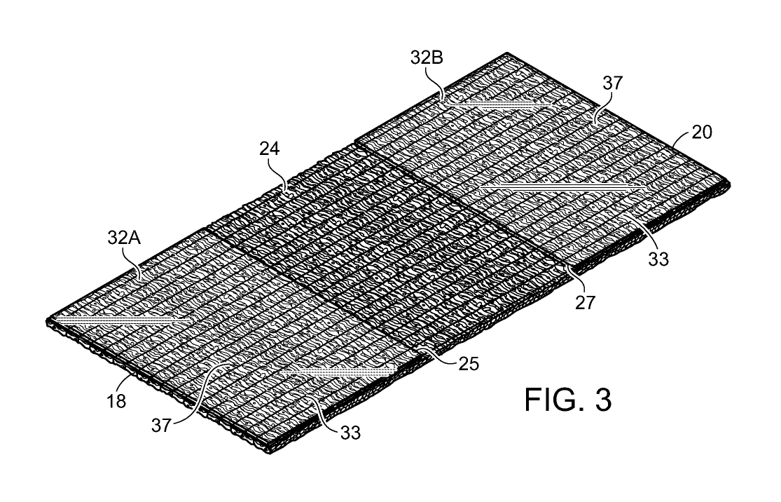

FIG. 2 shows the bottom side 16 of the securement wrap 10 including a protective tape 30 covering each of an adhesive portion 32A, 32B of the securement wrap (See FIG. 3). As shown in FIGS, 2 and 3, the securement wrap 10 includes an adhesive portion 32A extending along a length L of the wrap portion 12 from a first outer edge 25 of the central area 24 outwardly to the first end 18 of the wrap portion 12. Similarly, a second adhesive portion 32B extends along the length L of the wrap portion 12 from a second outer edge 27 of the central area 24 to the second end 20 of the wrap portion. The first and second outer edges 25, 27 of the central area 24 being located on opposite sides (edges) of central area and disposed generally orthogonal relative to the length L of wrap portion 12.

The material 26 is formed of a plurality of spaced apart elastic yarns 29 disposed parallel one to the other and extending throughout the length L of the wrap portion 12. A fibrous woven or not woven web 28 is attached to, and between the yarns 29. The web 28 being attached to the yarns 29 while the yarns are in a stretched position so the finished elastic material 26 remains shirred in a normal state. The material 26 including the yarns 29 and the webs 28 may be coated or impregnated with a polymer binder. Preferably the polymer binder is cohesive so that the elastic material 26 is coherent but will not adhere to clothing, hair, skin, or the catheters or other devices mentioned herein.

in the FIG. 3 embodiment, the adhesive portions 32A, 32B may comprise two-sided adhesive tape 33 having a first side of the adhesive tape fixed to the bottom side 16 of the wrap portion 12. The second side of the tape 37 being exposed and available to be removably adhere to a patient's skin. The adhesive portions 32A, 32B being spaced apart on either side of the central area 24 and extending a full width W of the wrap portion 12. The adhesive tape 33 may be flexible and conformable to a body part for attachment thereto. In one embodiment, the adhesive tape 33 may include a silicone gel adhesive tape consisting of a high adhesion silicone gel skin contact adhesive coated on a polyurethane carrier. The silicone gel adhesive tape provides instantaneous tack for adhering to the skin while being removable for discarding or repositioning the securement wrap 10 on the skin 35. The silicone gel adhesive tape may be hypoallergenic.

In other embodiments, the adhesive tape 33 may include different adhesives, one type of adhesive for attaching the adhesive tape 33 to the wrap portion 12 and another type of skin adhesive for attaching the tape 33 to the skin 35 of the patient. The adhesive for attaching the second side of the tape 37 to the skin 35 of a patient may be a different type of skin compatible adhesive than the silicone gel adhesive tape described hereinabove.

In another embodiment, a securement wrap 10 includes the adhesive portions 32A, 32B comprising an adhesive applied directly to the bottom side 16 of the wrap portion 12. Thus, the wrap portion 12 provides a backing for the adhesive and a separate carrier for the adhesive is not necessary.

As shown in FIG. 2, the central area 24 of the wrap portion 12 extends through the full width W of the wrap portion and defines a length approximately equal to one-third the length L of the wrap portion so that a length of the central area 24 is approximately equal to a length of each of the adhesive portions 32A, 32B. In other embodiments, a length of the central area 24 may be less than a length of the adhesive portions 32A and 32B. For example, if the securement wrap 10 is designed for use with a catheter of small diameter, the central area 24 may have a length shorter than the length of the adhesive portions 32A and 32B. In other embodiments, the securement wrap 10 may include a central area 24 having a length greater than the length of one or both of the adhesive portions 32A, 32B. For example, if the securement wrap 10 is configured to secure a monitoring device (not shown) or other object to a person's body, a length of the central area 24 may be longer than a length of the adhesive portions 32A and/or 32B. A longer central area 24 may be beneficial to securing objects having a cross-section larger than a catheter or other tubular device to one's body.

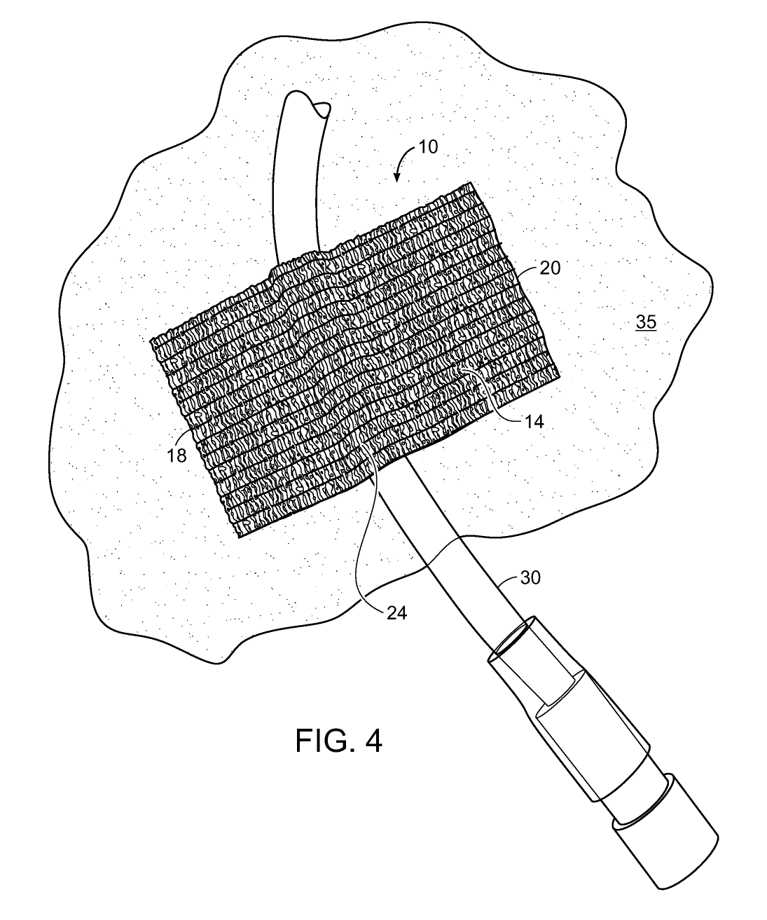

Referring to FIG. 4, the securement wrap 10 is shown as used for securing a portion of a catheter 30 against the skin 35 of a patient. The securement wrap 10 includes the adhesive portions 32A, 32B (not shown) adhered to the skin 35 of the patient. The central area 24 is wrapped tightly over the catheter 30 fixing a position of the catheter against the skin 35 of the patient. As is obvious to one skilled in the art, the protective tapes 30 have been removed from the adhesive portions 32A, 32B, prior to attaching the adhesive portions including the second side of the adhesive tape 37 to the skin 35. As shown in FIG. 4, the central portion 24 flexibly conforms to the shape of the catheter and fixes the catheter 30 securely against the patient's skin 35. The central area 24 of the wrap portion 12 does not adhere to the catheter 30 and is therefore easily removable from the catheter. Upon removal of the central area 24 from the catheter 30, no residue from the polymer binder and the securement wrap 10 remains on the catheter. Thus the securement wrap 10 provides improvements over prior art tapes wherein sticky residue often may remain attached to the catheter which collects dust and dirt and is often difficult to remove from the catheter or other device.

Adhesive residue which is often left from prior art securement tapes on catheters and other devices is a familiar and longstanding problem and inconvenience for medical patients. The presently disclosed securement wrap 10 resolves this longstanding problem by providing the claimed securement wrap to fix the catheter 30 to the skin 35 via the central area 24 of the wrap portion 12 disposed between the adhesive portions 32A, 32B, without the central area being adhered to the catheter. Thus, no adhesive residue is transferred to the catheter 30 or remains on the catheter once the securement wrap 10 is removed from the patient's skin 35.

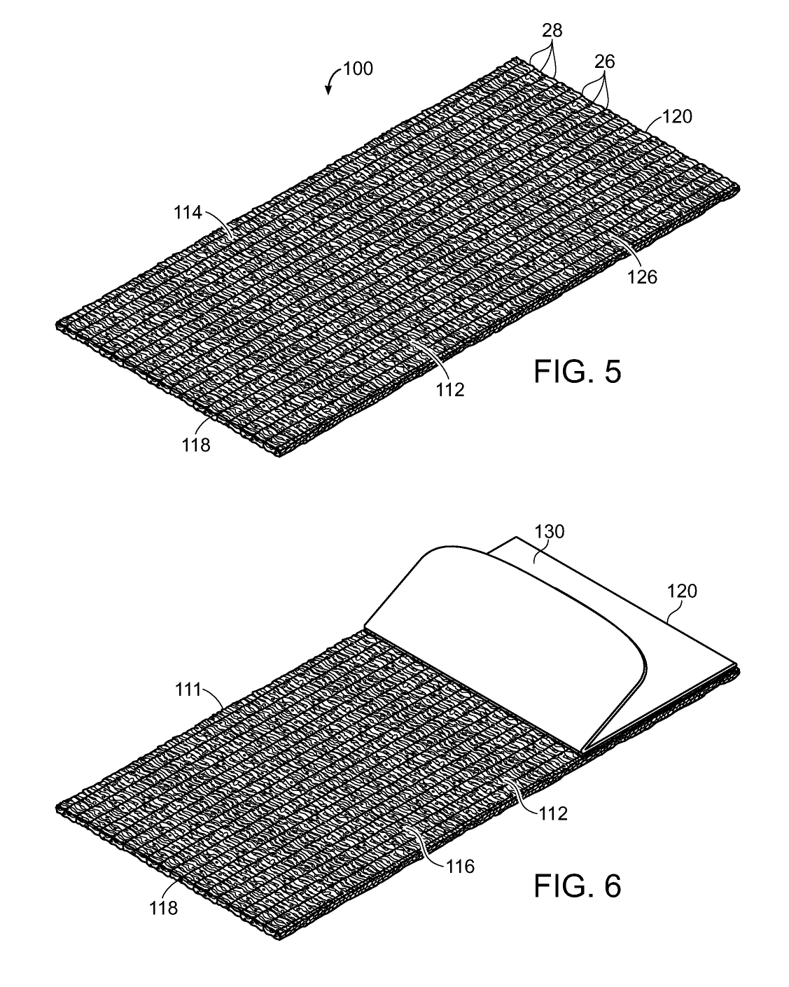

FIGS. 5-8 show a securement wrap 100 according to another embodiment of the present disclosure. The securement wrap 100 is similar to the securement wrap 10 described herein above and includes many of the same elements as set forth above. In the various figure, like elements are referred to with the same or similar reference numbers. The securement wrap 100 includes a wrap portion 112 having a top side 114, a bottom side 116, a first end 118, a second end 120. The wrap portion 112 is formed of an elastic self-adherent material 126. The material 126 being flexible and conformable to a body part or other object and being the same as the material 26 described above with respect to the securement wrap 10.

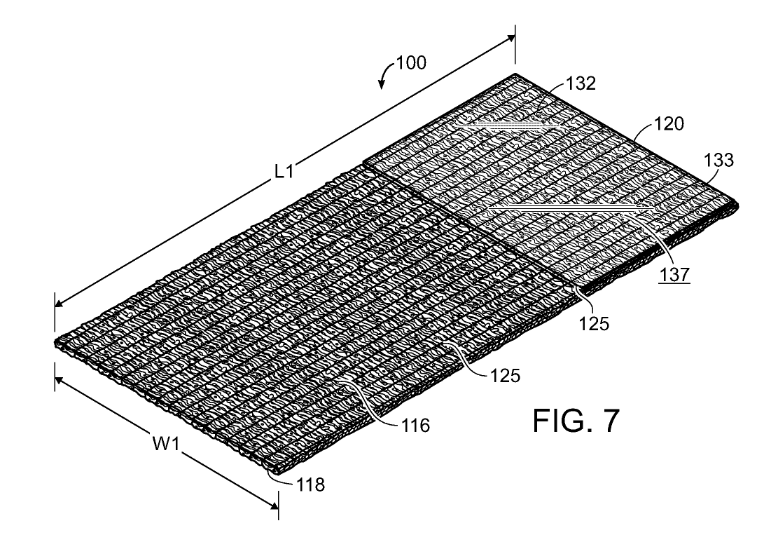

FIGS. 6 and 7 show the bottom side 116 of the securement wrap 100 including a protective tape 130 covering an adhesive portion 132 (See FIG. 7). The securement wrap 100 includes an adhesive portion 132 extending along a length L1 of the wrap portion 112 from an inside edge 125 of the adhesive portion 32 outwardly to the second end 120 of the wrap portion 112. The securement wrap of FIGS. 5-8 is similar to the securement wrap 10 of FIGS. 1-4 but includes only one adhesive portion 132 disposed adjacent one end 120 of the wrap portion 112. A free end 111 of the wrap portion 112 extends between the adhesive portion 132 to the second end 120 of the wrap portion.

FIG. 7 shows the bottom side 116 of the securement wrap 100, including the adhesive portion 132 comprising an adhesive tape 133 having a first side of the adhesive tape fixed to the bottom side 116 of the wrap portion 112. The second side 137 of the tape 133 being exposed and available to be removably adhered to a patient's skin. The adhesive portion 132 extending a full width W1 of the wrap portion 112. The adhesive tape 133 being flexible and conformable to a body part for attachment thereto.

As shown in FIG. 7, the securement wrap 100 includes the adhesive portion 132 at only a second end 120 of the wrap portion 112. The free end portion 111 of the securement wrap 100 with no adhesive, extends from an inside edge 125 of the adhesive portion 132 to the opposing first end 118 of the wrap portion 112.

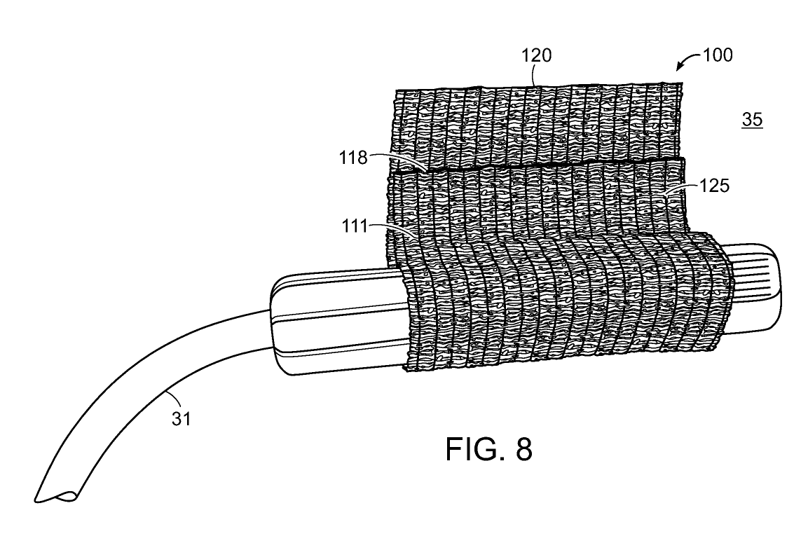

FIG. 8 shows the securement wrap 100 as used to attach a catheter 31 to the skin 35 of a patient. As shown, the second end 120 of the securement wrap is adhered to the skin 35 via the adhesive portion 132 (not visible in FIG. 8) of the wrap portion 112. The free end 111 is shown wrapped snugly about the catheter and attached with the first end 118 of the wrap portion coupled to the wrap portion 112 near the second end 120 thereof in a self-adherant connection. The bottom side 116 of the first end 118 of the wrap portion is adhered to the top side 114 of the wrap portion near the second end thereof. Thus, the catheter is securely attached to the patient's body and skin 35 without any adhesive or tape residue contacting the catheter 31. The attachment process can include wrapping the free end 111 of the wrap portion 112 about the catheter 31 and then attach the adhesive portion 132 to the skin 35 of the patient. Alternatively, the adhesive portion 132 can be attached to the skin 35 of the patient first, and then the catheter 31 can be wrapped with the free end 111 of the wrap portion, with a portion of the free end 111 secured to the wrap portion 112 near the second end 120 thereof. The free end 11 secured about the catheter 31 and conforming to the surface thereof thereby securely attaching the catheter to the skin 35 of the patient.

As will be obvious to one skilled in the art, one can release the catheter 31 from the fixed position attached to the skin 35 by releasing the self-adherent first and second ends 118, 120 respectively, of the wrap portion 112 from one another. The wrap portion 112 can be removed from the skin 35 by peeling the adhesive portion 132 from the skin 35.

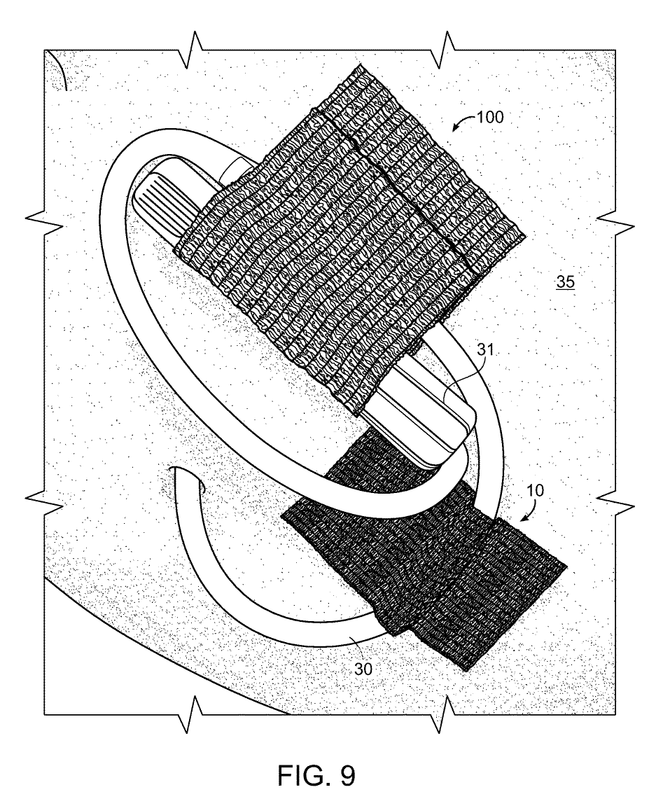

FIG. 9 illustrates the securement wraps 10 and 100 shown as used together to attach a catheter 30, 31 to the skin 35 of a patient in the same manner as discussed hereinabove with respect to FIGS. 4 and 8.

Example embodiments and methods thus being described, it will be appreciated by one skilled in the art that example embodiments may be varied through routine experimentation and without further inventive activity. Variations are not to be regarded as departure from the spirit and scope of the exemplary embodiments, and all such modifications as would be obvious to one skilled in the art are intended to be included within the scope of the following claims.

What is claimed is:

1. A securement wrap for a catheter and other devices comprising:
a wrap portion having a top side, a bottom side, a first end, a second end, and a central area located between the first and second ends; the wrap portion formed of an elastic self-adherent material;
an adhesive layer comprising first and second adhesive portions disposed at each of the first and second ends of the bottom side of the wrap portion, respectively; the first and second adhesive portions for attaching the respective first and second ends of the wrap portion to the skin of a person;
the central portion being releasable attachable to a catheter or other device for securing the catheter or other device to the body of a person.

2. The securement wrap in accordance with claim 1 wherein a length of the central portion is approximately equal to a length of each of the first and second adhesive portions.

3. The securement wrap in accordance with claim I wherein the first and second adhesive portions extend across a full width of the wrap portion.

4. The securement wrap in accordance with claim 1 wherein the wrap portion is configured to wrap about a catheter or other device and conform to a shape thereof.

5. The securement wrap in accordance with claim 1 wherein the wrap portion comprises a plurality of spaced apart elastic yarns disposed between a pair of coextensive non-woven fibrous webs.

6. The securement wrap in accordance with claim 5 wherein the plurality of elastic yarns extend along a length of the wrap portion.

7. The securement wrap in accordance with claim 1 wherein the first and second adhesive layers comprise adhesive applied to the bottom side of the wrap portion.

8. The securement wrap in accordance with claim 1 further comprising a protective tape attached to each of the first and second adhesive port 0

9. The securement wrap in accordance with claim 1 wherein the first and second adhesive portions comprise a two-sided tape attached to a bottom side of the wrap portion.

10. The securement wrap in accordance with claim 1 wherein the wrap portion does not adhere to hair, skin, clothing or plastics.

11. A securement wrap for a catheter and other devices comprising:
a wrap portion having a top side, a bottom side, a first end, and a second end, the wrap portion formed of an elastic self-adherent material;
an adhesive portion disposed at the first end of the bottom side of the wrap portion; the adhesive portion for attaching the first end of the wrap portion to the skin of a person;
a free end of the wrap portion extending from the adhesive portion to the second end of the wrap portion;
the free end being releasable attachable to a catheter or other device for securing the catheter or other device to the body of a person.

12. The securement wrap according to claim 11 wherein the free end is configured to encircle the catheter or other device and attach to the wrap portion near the first end thereof.

13. The securement wrap in accordance with claim 11 wherein the adhesive portion extends across a full width of the wrap portion.

14. The securement wrap in accordance with claim 11 wherein the free end is configured to wrap about a catheter or other device and conform to a shape thereof.

15. The securement wrap in accordance with claim 11 wherein the adhesive portion comprises adhesive applied to the bottom side of the wrap portion.

16. The securement wrap in accordance with claim 11 wherein the wrap portion comprises a shirred material,

17. The securement wrap in accordance with claim 11 wherein the wrap portion does not adhere to hair, skin, clothing or plastics.
